SEQUENCE LISTING (1 sequence)

SEQ ID NO: 1 - protein, 552 aa, Homo sapiens
MSTFGYRRGLSKYESIDEDELLASLSAEELKELERELEDIEPDRNLPVGLRQKSLTEKTPTGTFSREALMAYWEKESQKLLEKERLGECGKVAEDKEESEEELIFTESNSEVSEEVYTEEEEEESQEEEEEEDSDEEERTIETAKGINGTVNYDSVNSDNSKPKIFKSQIENINLTNGSNGRNTESPAAIHPCGNPTVIEDALDKIKSNDPDTTEVNLNNIENITTQTLTRFAEALKDNTVVKTFSLANTHADDSAAMAIAEMLKANEHITNVNVESNFITGKGILAIMRALQHNTVLTELRFHNQRHIMGSQVEMEIVKLLKENTTLLRLGYHFELPGPRMSMTSILTRNMDKQRQKRLQEQKQQEGYDGGPNLRTKVWQRGTPSSSPYVSPRHSPWSSPKLPKKVQTVRSRPLSPVATLPPPPPPPPPPPPSSQRLPPPPPPPPPPLPEKKLITRNIAEVIKQQESAQRALQNGQKKKKGKKVKKQPNSILKEIKNSLRSVQEKKMEDSSRPSTPQRSAHENLMEAIRGSSIKQLKRVEVPEALRWEHDL

(19) United States
(12) Patent Application Publication
(10) Pub. No.: US 2003/0170890 A1
(43) Pub. Date: Sep. 11, 2003

(54) Pathologically modified myocardial cell, production and use thereof

(76) Inventors: Volker Rönicke, (US); Barbara Nave, M?uuml;nchen (DE); Thomas Henkel, M?uuml;nchen (DE)

Correspondence Address: CLARK & ELBING LLP, 101 FEDERAL STREET, BOSTON, MA, 02110, US

(21) Appl. No.: 10/168,235

(22) PCT Filed: Dec. 21, 2000
(86) PCT No.: PCT/EP00/13101

(30) Foreign Application Priority Data
Dec. 22, 1999 (DE) 19962154.3

(51) Int. Cl. C12N 5/08; C12N 15/85; C12Q 1/00

(52) U.S. Cl. 435/366; 435/4; 435/455

(57) ABSTRACT

Pathologically modified myocardial cell which can be produced from healthy cardiac tissue by provision or isolation of at least one healthy myocardial cell, stimulation of the isolated myocardial cell by suitable hormones, hormone analogs and/or cytokines; detection of the at least one pathologically modified myocardial cell by determination of the localization of at least one signal molecule, and methods for the production thereof and the use thereof including a method for detecting or identifying substances acting on the heart.

The present invention relates to a pathologically modified myocardial cell which can be produced from healthy cardiac tissue by isolation of at least one healthy myocardial cell, stimulation of the isolated myocardial cell by suitable hormones, hormone analogs and/or cytokines; and detection of the at least one pathologically modified myocardial cell through determination of the localization of at least one signal molecule, preferably of at least one protein in the sarcomere. The invention additionally relates to a method for producing a pathologically modified myocardial cell, a method for detecting or for identifying substances acting on the heart, and the use of a pathologically modified myocardial cell.

Besides the heart as the central element, the cardiovascular system consists of large and intermediate vessels with a defined arrangement, and many small and very small vessels which arise and regress as required. The cardiovascular system is subject to self-regulation (homeostasis) whereby peripheral tissues are supplied with oxygen and nutrients, and metabolites are transported away. The heart is a muscular hollow organ with the task of maintaining, through alternate contraction (systole) and relaxation (diastole) of atria and ventricles, the continuous blood flow through vessels.

The muscle of the heart, the myocardium, is a functional assemblage of cells (syncytium) which is composed of striated muscle cells and is embedded in connective tissue. Each cell has a nucleus and is bounded by the plasma membrane, the sarcolemma. The contractile substance of the heart is formed by highly organized, long and parallel cellular constituents, the myofibrils, which in turn are separated irregularly by sarcoplasm. Each myofibril is divided into a plurality of identical structural and functional units, the sarcomeres. The sarcomeres in turn are composed of the thin filaments, which mainly consist of actin, tropomyosin and troponin, and the thick filaments, which mainly consist of myosin. The center of each sarcomere is referred to as the M-line, where thick filaments of opposite orientation meet one another. The sarcomere is bounded by the Z-bands which ensure the anchorage of the thin filaments and represent the connection to the next sarcomere.

The molecular mechanism of muscle contraction is based on a cyclic attachment and detachment of the globular myosin heads by the actin filaments. On electrical stimulation of the myocardium, Ca²⁺ is released from the sarcoplasmic reticulum, which influences, through an allosteric reaction, the troponin complex and tropomyosin and, in this way, permits contact of the actin filament with the myosin head. The attachment brings about a conformational change in the myosin which then pulls the actin filament along itself. ATP is required to reverse the conformational change and to return to the start of a contraction cycle.

The activity of the myocardium can be adapted by nervous and hormonal regulatory mechanisms in the short term to the particular blood flow requirement (perfusion requirement). Thus, both the force of contraction and the rate of contraction can be increased. If the strain is prolonged, the myocardium undergoes physiological reorganization mainly characterized by an increase in myofibrils (myocyte hypertrophy).

If the myocardium is damaged, the originally physiological adaptation mechanisms frequently lead in the long term to pathophysiological states, resulting in chronic heart failure (cardiac insufficiency) and usually ending with acute heart failure. If the insufficiency is severe and chronic, the heart is no longer able to respond appropriately to changed output demands, and even minor physical activities lead to exhaustion and shortness of breath.

Damage to the myocardium results from deprivation of blood (ischemia) which in turn is caused by cardiac disorders, bacterial or viral infections, toxins, metabolic abnormalities, autoimmune diseases or genetic defects. Therapeutic measures at present aim at strengthening the force of contraction and controlling the compensatory neuronal and hormonal compensation mechanisms. Despite this treatment, the mortality rate after diagnosis of cardiac insufficiency is still high (35 to 50% within the first five years after diagnosis). It is the main cause of death around the world. The only causal therapy applied at present is the cost-intensive heart transplant, which is associated with considerable risks for the patient.

In order to develop novel causal therapies it is necessary to understand in detail the cellular reorganization of the myocardial cells (cardiomyocytes) which is associated with the development and progression of a myocardial disorder. It is known at present, from cell culture experiments with HeLa, HEK 293 or CHO cells, that external signals are picked up by cellular receptors and transmitted via signal transduction pathways or networks or cascades into the interior of the cell. The activation of receptors by signal molecules results in the initiation of intracellular enzyme cascades which regulate the Ca²⁺ balance, the energy status of the cell, gene expression and protein biosynthesis.

In order to investigate the specific signal transduction in myocardial cells and elucidate their effect on heart diseases, mainly neonatal rat cardiomyocytes have been used. It has been possible with the aid of this model system to identify several signal transduction pathways in myocardial cells, in which at least four different receptor classes are important:

i) G-protein-coupled receptors, such as adrenergic receptors or endothelin receptors;

ii) receptor tyrosine kinases, such as IGF-1 receptors;

iii) cytokine receptors, such as receptors for cytokines of the interleukin-6 family and

iv) serine/threonine receptor kinases, such as TGF-β receptors.

re i) The first group of receptors are G-protein-coupled receptors, which include adrenergic receptors. The adrenergic receptors are differentiated into α₁, α₂ and β types, with each type in turn comprising three subtypes. Whereas all β-adrenergic receptors increase the concentration of cyclic adenosine monophosphate (cAMP) via the Gαₛ subunit of the trimeric G-proteins, the α-adrenergic receptors activate various G-protein components which are in turn able to reduce the cAMP content (Selbie and Hill, (1998) Trends. Pharmacol. Sci. 19, p. 87). An increased cAMP concentration activates protein kinase A (PKA) which is in turn involved inter alia in the regulation of the Ca²⁺ balance (Hefti et al. (1997) J. Mol. Cell. Cardiol. 29, p. 2873). Isoforms of protein kinase C (PKC) can also be activated via this pathway (Castellano and Böhm (1997) Hypertension 29, p. 715). It was further possible to show that PKC is an activator of the raf-MAP kinase cascade and, in cell culture systems, stimulates both cell growth and cell division (Ho et al. (1998) JBC 273, p. 21730).

The endothelin receptors likewise belong to the G-protein-coupled receptors and occur as the ET_{A} and ET_{B} types, at least some of which perform different tasks (Miyauchi and Masaki (1999) Ann. Rev. Physiol. 61, p. 391). The ET_{A} and ET_{B} receptors can be stimulated by the signal molecule ET-1, which also leads to activation of phospholipase Cγ (PLCγ). Activated PLCγ subsequently catalyzes the conversion of phosphatidylinositiol 4,5-bisphosphate (PIP₂) into diacylglycerol (DAG) and inositol triphosphate (InsP₃) (Dorn et al. (1999) Trends Cardiovasc. Med. 9, p. 26). DAG in turn activates isoforms of the PKC family, whereas InsP₃ causes the release of Ca²⁺ from intracellular Ca²⁺ stores.

An increased Ca²⁺concentration in myocardial cells influences the contraction and activates further signal transduction proteins such as, for example, isoforms of PKC (Nakamura and Nishizuka (1994), J. Biochem. 115, p. 1029).

re ii) Another important group in the transmission of cellular signals are the receptor tyrosine kinases which activate a number of signal transduction molecules such as, for example, the adaptor proteins Grb2, APS or She, which in turn have a positive influence on phosphatidylinositol 3-kinase or ras. The MAP kinase cascade is switched on by these activated proteins, leading to increased protein biosynthesis and cell growth (Ho et al. (1992) Cell 71, p. 335).

Within the MAP kinase cascade a distinction is made between three signal transduction pathways which are referred to as the ERK, p38 and JNK kinase signal transduction pathways. It is known from cell culture experiments that PKC mainly activates the ERK signal transduction pathway which promotes protein biosynthesis and cell division (Sugden et al. (1998) Adv. Enzyme Regul. 38, p. 87). The p38 signal transduction pathway by contrast is thought to be connected with programmed cell death (apoptosis) and can be induced in the cell by endotoxins, cytokines and physiological stress (Wang et al. (1998) JBC, 273, p. 2161). The JNK kinase signal transduction pathway is likewise induced by stress factors, with the activation proceeding via PKC, MAP-ERK kinases (MEKK) and Sek kinases and likewise leading to increased gene transcription (Lazou (1998) J. Biochem. 332, p. 459).

re iii) The third group of receptors, which are embraced by the term cytokine receptors, are distinguished by the particular feature that they do not contain their own kinase activity. The cytokine receptors include the LIF receptor which in turn is assigned to the interleukin-6 family. The LIF receptor is composed of a ligand-specific component and of a GP130 subunit. GP130 in the activated state brings about a signal transduction which attracts JAK and Tyr kinases. These kinases phosphorylate STAT proteins (signal transducer and activator of transcription) which are thus prepared for entry into the cell nucleus. There the STAT proteins influence gene expression (summarized in: Tetsuya Taga (1997) Ann. Rev. Immunol. 15, pp. 797-819 “GP 130 and the Interleukin-6 Family of Cytokines”).

re iv) The last group of receptors, the serine/threonine receptor kinases, has received increased attention only recently. It includes the TGF-β receptor which transmits extracellular signals to intracellular SMAD proteins which in turn are phosphorylated. After phosphorylation, the SMAD proteins migrate actively into the cell nucleus, there bind to DNA and specifically activate gene transcription (Attisano et al. (1998) Curr. Opin. Cell. Biol. 10, p. 188).

Many of the mediators involved in the signal transduction pathways and the relations between the pathways and mediators are now known. On the basis of these results, initial studies have been undertaken in order to be able to make statements about the pathologically modified heart.

Thus, for example, test systems for determining the degree of hypertrophy of myocardial cells which are essentially based on measurement of an altered expression of particular genes, of the increase in general protein biosynthesis or on measurement of the performance of the heart (morphology) are known. The experimental approaches have the serious disadvantage that they take no account of signal transduction pathways which may in the diseased heart be specifically up- or downregulated compared with the healthy heart.

The parameter used most often for determining the condition of the myocardial cell in the known experimental approaches is the increase in ANP expression (atrial natriuretic peptide), although the functional connection between an increase in ANP and hypertrophy has not to date been explained. In addition, the increase in the expression rate of transcription factors such as c-fos, c-jun or erg-1 are used for describing a hypertrophy of myocardial cells. The third group of genes showing increased expression during hypertrophy are structural components of the contractile apparatus, the direct connection with the development of hypertrophy being unclear in all cases (Lowes B. D. et al. (1997) J. Clin. Invest. 100, pp. 2315-2324; Shubeita H. E. et al. (1990) JBC 265, 33, pp. 20555-62; Iwaki K. et al. (1990) JBC 265, 23, pp. 13809-17; Donath et al. (1994) Proc. Natl. Acad. Sci., USA, 91, pp. 1686-1690).

The increased expression of components of the contractile apparatus makes an essential contribution to the increase in the total protein synthesis rate, which results in a measurable increase in the volume of the myocardial cells. It is used as further indicator of hypertrophy and either measured as increase in the surface area after fixation and staining of the cells or assessed through determination of the ratio of the changes in the length and width of the cells (U.S. Pat. No. 5,837,241; Wollert K. C. (1996) JBC 271, 16, pp. 9535-45).

None of the described methods is suitable for simulating the human in vivo situation in vitro because the unambiguous correlation between the increased expression rate of individual genes and the hypertrophy of myocardial cells is not explained.

The present invention is thus based on the object of providing a pathologically modified myocardial cell with the aid of which it is possible to investigate the molecular changes leading to heart diseases in vivo and with the aid of which it is possible to find substances for their efficacy for the prophylaxis and therapy of cardiac patients.

It has now been found, surprisingly, that stimulation of neonatal rat cardiomyocytes with hormones, hormone analogs and/or cytokines in cell culture leads to an altered localization, compared with unstimulated cardiomyocytes, of at least one signal molecule in the sarcomere of the myocardial cell. The gene of the signal molecule has been isolated from a cDNA bank of human cardiac tissue, and it was possible to show that there is stronger expression of this gene in insufficient cardiac tissue than in healthy cardiac tissue, suggesting a causal connection between this gene expression and the observed cardiac insufficiency. Because of its association with heart diseases associated with hypertrophy of myocardial cells, in particular dilated cardiomyophathy (DCM), the gene product of the signal molecule is referred to as DCMAG-1 protein. Its amino acid sequence is depicted in SEQ ID NO: 1. On stimulation of an isolated myocardial cell by suitable hormones, hormone analogs and/or cytokines, the DCMAG-1 gene product can be detected specifically in the sarcomere of the myocardial cells, whereas it is uniformly distributed in the cytoplasm in the unstimulated myocardial cell. This difference in the subcellular localization of the DCMAG-1 gene product is also detectable in heart biopsies from DCM patients compared with healthy people. In addition, the same shift in localization of the DCMAG-1 gene product was inducible in an animal experiment in a DCM induced by increased rate of contraction.

In the diseased heart therefore it is possible to use the increasing association of the DCMAG-1 gene product with the Z-band as criterion for progression of the course of the disorder. This shift, associated with a reorganization of the Z-band during heart diseases, in the localization of the DCMAG-1 gene product is so surprising because the structure of the Z-band has to date been regarded as static and therefore has received little attention (Alexander R. W. et al. (1997) in Hurst's “The Heart”, 9th Edit., McGraw Hill, p. 74). In addition, to date, only reorganization of the complete sarcomeres from a parallel to a serial arrangement has been perceived, so that it was not possible to suspect an association between a shift of the localization of particular gene products which are expressed more strongly in the diseased heart, and heart diseases such as DCM.

One aspect of the invention is therefore a pathologically modified myocardial cell which can be produced from healthy cardiac tissue and/or at least one healthy myocardial cell by a method comprising the steps:

(a) provision or isolation of at least one healthy myocardial cell;

(b) stimulation of the isolated myocardial cell by suitable hormones, hormone analogs and/or cytokines.

The terms “healthy cardiac tissue or healthy myocardial cell” mean for the purpose of the present invention cardiac tissues or cells isolated therefrom which are clinically unremarkable. The myocardial cells were isolated from biopsy material whose donors showed no signs of chronic cardiac insufficiency associated with hypertrophy of myocardial cells. A further possibility is to obtain a healthy myocardial cell by in vitro differentiation from stem cells. Methods of this type are described, for example by Kolossov E. et al. (1998) J Cell Biol 28; 143(7), pp. 2045-2056.

Accordingly, the term “pathologically modified myocardial cell” means for the purpose of the present invention a myocardial cell which has been isolated from biopsy material of a patient with heart disease, for example insufficiency. This term additionally means a myocardial cell which has been stimulated according to the invention and has the histopathological appearance of such a pathological myocardial cell. This can be achieved by in vitro stimulation of the myocardial cells, which thus show a shift in the localization of particular signal molecules from the cytoplasm into the sarcomere, for example into the M-line or into the Z-band. This shift is like that evident in myocardial cells obtained from the hearts of patients with insufficiency.

Accordingly, the term “signal molecule” means for the purpose of the present invention a cellular, endogenous molecule or protein which occurs in particular in myocardial cells and which, after hormone, hormone analog and/or cytokine stimulation, changes its localization within the myocardial cell compared with the healthy starting cell. In this connection, “signal molecule” means in particular a protein of the sarcomere of myocardial cells.

The term “suitable” hormones means for the purpose of the present invention in particular the hormones epinephrine, norepinephrine including their derivatives, and ET-1, ET-2, ET-3, angiotensin I and II, insulin (IN), IGF-1 and myotrophin. The “suitable” hormone analogs which are preferably used are catecholamine derivatives such as, for example, isoproterenol (ISO) and phenylephrine (PE). “Suitable” cytokines mean for the purpose of the present invention in particular LIF, cardiotrophin-1 (CT-1), interleukin-6 and -11 (IL-6 and -11), oncostatin M and ciliary neurotrophic factor.

The healthy starting material for producing the pathologically modified myocardial cell may be derived from birds, in particular from chickens, or from mammals. In the case of mammals, particular preference is given to human cardiac tissue, and cardiac tissue from rabbits and rodents, in the latter case in particular from rats.

The stimulation of the myocardial cell takes place with the described hormones, hormone analogs and/or cytokines essentially simultaneously. Thus, various stimulants can be mixed together, whereby their use takes place absolutely simultaneously. “Essentially simultaneous” stimulation likewise means use of the various stimulants in immediate succession.

The hormones, hormone analogs and/or cytokines act via signal transduction cascades which have already been described under (i) to (iv) and at least some of which are different, in particular via various receptors on or in the myocardial cell.

In a further preferred embodiment, said hormones, hormone analogs and/or cytokines activate signal transduction cascades, not via receptors but by acting directly on cascades subject to the receptors. Such a stimulation can be effected for example by phorbol esters such as phorbol myristate acetate (PMA). Thus, it is known that phorbol ester is able to bind protein kinase C (PKC) directly and requires no receptor for this. The direct interaction activates the kinase activity of PKC, especially of the conventional PKC isoforms α, βI, βII and γ. The interaction between phorbol ester and PKC is very sensitive and can lead to significant PKC stimulation even with 1 nM phorbol ester (Gschwendt et al. (1991) TIBS, 16, p. 167). Stimulation of PKC by phorbol ester leads, just like receptor-mediated stimulation of PKC, to increased gene transcription, protein biosynthesis and cell growth.

A further aspect of the present invention is a method for producing the myocardial cell of the invention from healthy cardiac tissue and/or from at least one healthy myocardial cell, where the method comprises the following steps:

(i) provision or isolation of at least one healthy myocardial cell;

(ii) stimulation of the isolated myocardial cell by suitable hormones, hormone analogs and/or cytokines; and where appropriate

(iii) detection of the at least one pathologically modified myocardial cell by determination of the localization of at least one signal molecule, preferably at least one protein, in the sarcomere.

Detection of the localization of the signal molecule, which is preferably a protein, is preferably carried out at the single-cell level. The term “single-cell level” means for the purpose of the present invention for example the microscopic examination of a single cell in relation to specific properties. Morphological features of the cells such as their size or their shape may in this case contribute to the characterization. A particularly preferred method for examining signal molecules, in particular proteins, at the single-cell level is microscopic detection by means of immunofluorescence. In this method, proteins are detected by colocalization with known proteins within their cellular structure. This term also means the examination by electron microscopy of subcellular structures such as, for example, sarcomeres.

Thus, for example, the association of a sarcomere protein with known Z-band proteins such as α-actinin after stimulation can be identified as component of the Z-band by means of immunofluorescence. The DCMAG-1 gene product is particularly suitable because it has been possible to show in vitro and in vivo that it is uniformly distributed in the cytoplasm in unstimulated and healthy myocardial cells, whereas it is colocalized together with α-actinin in the Z-band in stimulated and pathological myocardial cells. However, in vitro it is possible to observe not only the Z-band localization but also a staining of the M-line. The DCMAG-1 gene product can be labeled for example by a specific antibody and detected by subsequent immunofluorescence using methods known to the skilled worker. A further immunological detection method for colocalization of proteins at the single-cell level is immunoelectron microscopy which is likewise known to the skilled worker.

It has further been possible to show in relation to rat myocardial cells that stimulation by phorbol ester brings about a shift in the DCMAG-1 gene product into the middle of the M-line of the sarcomere.

A further possibility for detecting proteins at the single-cell level is to use fusion proteins between, for example, the DCMAG-1 gene product and a marker protein. Examples of such marker proteins are prokaryotic peptide sequences which may be derived, for example, from the galactosidase of E. coli. A further possibility is to use viral peptide sequences, such as that of bacteriophage M13, in order in this way to generate fusion proteins for the phage display method known to the skilled worker (Winter et al. (1994) Ann. Rev. Immunol., 12, pp. 433-455). Likewise suitable as marker proteins are the so-called fluorescent proteins which are referred to, depending on the fluorescent color, as B-, C-, G-, R- or YFP (blue, cyano, green, red or yellow fluorescent protein). Fluorescent fusion proteins can be employed for example via the fluorescence resonance energy transfer (FRET) method also for detecting protein-protein interactions at the single-cell level.

A further method for detecting the shift of the localization of particular proteins at the single-cell level is the characteristic modification of sarcomere proteins, in particular M-line proteins or of Z-band proteins. In this case it is possible to use postranslational modifications such as phosphorylations on serine, threonine and/or tyrosine residues for the detection through the use of specific antibodies. For example, a phosphorylation and/or dephosphorylation of the DCMAG-1 gene product at particular serine, threonine and/or tyrosine residues may be responsible for the association and binding to Z-band proteins.

Comparison of the protein sequence of the DCMAG-1 gene product with a protein database revealed a certain sequence homology with the protein tropomodulin. Tropomodulin is known as a protein which in chicken cardiomyocytes has an effect on the development of the myofibrils and on the contractility of the cells (Gregorio et al. (1995) Nature 377, pp. 83-86). This protein binds firstly to tropomyosin and secondly to the actin filaments, but its own activity is not regulated. The DCMAG-1 gene product likewise has some structural features of tropomodulin, such as, for example, a tropomyosin binding domain. In contrast to tropomodulin, the DCMAG-1 gene product has additional structural features which indicate regulation of the activity of the protein by tyrosine kinases.

The term “functional variant” of the amino acid sequence of the DCMAG-1 gene product means for the purpose of the present invention proteins which are functionally related to the protein of the invention, i.e. can likewise be referred to as regulatable modulator of the contractility of myocardial cells, are expressed in striated muscle, preferably in the myocardium and there in particular in myocardial cells, have structural features of tropomodulin such as, for example, one or more tropomyosin binding domains and/or whose activity can be regulated by tyrosine kinases.

Examples of “functional variants” are the corresponding proteins derived from organisms other than humans, preferably from nonhuman mammals.

In the wider sense, this also means proteins having a sequence homology, in particular a sequence identity of about 50%, preferably of about 60%, in particular of about 70%, with the DCMAG-1 gene product having the amino acid sequence shown in SEQ ID NO: 1. These include, for example, polypeptides which are encoded by a nucleic acid which is isolated from non-heart-specific tissue, for example skeletal muscle tissue, but have the identified functions after expression in a heart-specific cell. These also include deletions of the polypeptide in the region of about 1-60, preferably of about 1-30, in particular of about 1-15, especially of about 1-5, amino acids. These also include moreover fusion proteins which comprise the protein described above, where the fusion proteins themselves already have the function of a regulatable modulator of the contractility of myocardial cells or can acquire the specific function only after elimination of the fusion portion.

“Functional variants” also include in particular fusion proteins with a portion of, in particular, non-heart-specific sequences of about 1-200, preferably about 1-150, in particular about 1-100, especially about 1-50, amino acids. Examples of non-heart-specific protein sequences are prokaryotic protein sequences which may be derived for example from the galactosidase of E. coli or from the DNA binding domain of a transcription factor for use in the two-hybrid system described hereinafter. A further example which may be mentioned of non-heart-specific protein sequences are viral peptide sequences for use in the phage display method which has already been mentioned.

The nucleic acid of the invention which codes for the protein of the invention is generally a DNA or RNA, preferably a DNA. A double-stranded DNA is generally preferred for expression of the relevant gene.

A further aspect of the present invention is a method for the detection or for the identification of one or more substances acting on the heart, characterized in that the method comprises the following steps:

(i) provision or isolation of at least one myocardial cell;

(ii) contacting of the myocardial cell with one or more test substances; and

(iii) detection or identification of one or more substances acting on the heart through determination of the localization of at least one signal molecule, preferably at least one protein in the sarcomere.

In a particularly preferred embodiment there is use of a myocardial cell of the invention which, through stimulation with suitable hormones, hormone analogs and/or cytokines, shows the clinical appearance of a pathologically modified myocardial cell.

The term “test substances” for the purpose of the present invention means those molecules, compounds and/or compositions and mixtures of substances which may interact with the myocardial cell of the invention under suitable conditions. Possible test substances are low molecular weight, organic or inorganic molecules or compounds, preferably molecules or compounds having a relative molecular mass of up to about 1 000, in particular of about 500. Test substances may also be expressible nucleic acids which are brought by infection or transfection by means of known vectors and/or methods into the myocardial cell. Examples of suitable vectors are viral vectors, in particular adenovirus, or nonviral vectors, in particular liposomes. Suitable methods are, for example, calcium phosphate transfection or electroporation. The term “expressible nucleic acid” means a nucleic acid which firstly consists of an open reading frame and secondly comprises cis-active sequences, for example a promoter or a polyadenylation signal, which ensure transcription of the nucleic acid and translation of the transcript.

Test substances may also comprise natural and synthetic peptides, for example peptides having a relative molecular mass of up to about 1 000, in particular up to about 500, and proteins, for example, proteins having a relative molecular mass of more than about 1 000, in particular more than about 10 000, or complexes thereof. The peptides may moreover be encoded by selected or random nucleic acids, which are preferably derived from gene banks or nucleic acid libraries, the peptides being obtained by natural or artificial expression of the sequences. Likewise covered by this are kinase inhibitors, phosphatase inhibitors and derivatives thereof. The test substances may because of their interaction either reduce/prevent or favor/bring about the shift in localization of the DCMAG-1 gene product after stimulation.

A further aspect of the present invention is the use of a pathologically modified myocardial cell, preferably of a pathologically modified myocardial cell of the invention, for the detection or for the identification of one or more substances acting on the heart.

A suitable test system for identifying test substances is based on the identification of functional interactions with the so-called two-hybrid system (Fields and Stemglanz, (1994), TIGS 10, pp. 286-292; Colas and Brent, (1998) TIBTECH 16, pp. 355-363). In this test, cells are transformed with expression vectors which express fusion proteins composed of the DCMAG-1 gene product and of a DNA binding domain of a transcription factor such as, for example, Gal4 or LexA. The transformed cells additionally comprise a reporter gene whose promoter carry binding sites for the corresponding DNA binding domain. It is possible by transformation of another expression vector which expresses a second fusion protein composed of a known or unknown polypeptide with an activation domain, for example of Gal4 or herpes virus VP 16, to greatly increase the expression of the reporter gene if the second fusion protein functionally interacts with the polypeptide of the invention. This increase in expression can be utilized in order to identify novel interactors, for example by producing for the construction of the second fusion protein a cDNA library which codes for interactors of interest.

In addition, this test system can be utilized for screening substances which inhibit an interaction between the polypeptide of the invention and a functional interactor. Such substances reduce the expression of the reporter gene in cells which express the fusion proteins of the polypeptide of the invention and of the interactor (Vidal and Endoh, (1999), TIBS 17, pp. 374-81). It is thus possible rapidly to identify or detect novel substances which act on the heart and which may be both toxic and pharmaceutically effective.

Priority application DE 199 62 154.3, filed Dec. 22, 1999 including the specification, drawings, claims and abstract, is hereby incorporated by reference. All publications cited herein are incorporated in their entireties by reference.

The figures and the following examples are intended to explain the invention in more detail without restricting it.

### DESCRIPTION OF THE FIGURES

SEQ ID NO: 1 shows the amino acid sequence of the DCMAG-1 protein.

FIG. 1 shows an immunofluorescence of unstimulated neonatal rat cardiomyocytes which have been stained with a polyclonal anti-DCMAG-1 antibody and with a Cy3-coupled secondary antibody.

FIG. 2 shows an immunofluorescence of ET-1/ISO/LIF-stimluated neonatal rat cardiomyocytes which have been stained with a polyclonal anti-DCMAG-1 antibody and with a Cy3-coupled secondary antibody.

### EXAMPLES

1. Localization of DCMAG-1 in Healthy and Diseased Human Myocardium

Human cardiac tissue from donor hearts unsuitable for transplantation and explanted diseased patients' hearts (DCM) was deep-frozen at −80° C. immediately after explantation. Cryostat sections with a thickness of 4 μm were prepared from 5 different DCM hearts and 5 different healthy donor hearts. The histological sections were fixed with 3% paraformaldehyde solution and then incubated with monoclonal antibodies against α-actinin or with polyclonal anti-DCMAG-1 antibodies, the incubation with antibodies being referred to hereinafter as (antibody) staining (as described in Example 3). The evaluation was carried out under a fluorescence microscope (Axiovert 100S, Cy3 filter set, Zeiss, Gottingen).

The α-actinin staining of the healthy and of the DCM heart shows a pattern with sharp striations which is typical of a Z-band protein and is striated transverse to the course of the myofibrils. Whereas the DCMAG-1 staining of the healthy heart shows a uniform, diffuse staining of the sarcoplasm, a transversely striated pattern which correlates with the staining for a-actinin is evident for the DCM heart. This shows that, on comparison of healthy and DCM hearts, the DCMAG-1 protein changes its intracellular localization and migrates from the sarcoplasm into the Z-band, so that a molecular transformation of the Z-band takes place in connection with the pathological condition of DCM.

2. Generation of a Cardiac Pacemaker-Induced Cardiac Insufficiency in Rabbits

Chinchilla cross rabbits (2.5-3 kg) were kept under normal housing conditions and were permitted to drink and eat ad libitum. For the pacemaker implantation, the experimental animals were preinjected with medetomidine (10 μg/kg) and then anesthetized with propofol (5 mg/kg/h). Fentanyl (10 μg/kg) was administered intravenously for analgesia. The rabbits underwent controlled ventilation, and the blood pressure, the ECG and the blood oxygenation were continuously monitored.

Under sterile operating conditions, a 2 Fr pacemaker probe (Medtronic, Unterschleiβheim) was advanced via the right external jugular vein into the right ventricular cavity and was anchored. The pacemaker probe was then exteriorized subcutaneously via a needle to a previously made laterodorsal subcutaneous pocket and there connected to the cardiac pacemaker unit (Diamond II, Vitatron, Leiden, Holland, with user-defined software). The skin incisions were closed with surgical suture material. Cardiac stimulation was started with 320 heartbeats/min one week after pacemaker implantation. The pacemaker rate was increased by 20 beats/min each week. In addition, to monitor the development of cardiac insufficiency, the left ventricular fractional shortening was measured by echocardiography. After controlled pacing for three weeks, the experimental animals were sacrificed and the hearts were sectioned in a cryostat (thickness 4 μm) for histological examination.

The histological sections of the hearts were fixed with 3% paraformaldehyde. The antibody stains (α-actinin and DCMAG-1) took place as described in Example 3. The evaluation was carried out under a fluorescence microscope.

Comparison of the subcellular localization of DCMAG-1 on the basis of histological sections of the hearts shows a diffuse sarcoplasmic staining in the control rabbits, whereas a distinct transverse striation of the myocytes is evident in the rabbit with the induced cardiac insufficiency. This transverse striation is likewise shown with an α-actinin stain, so that DCMAG-1 associates with the Z-bands in hearts with cardiac insufficiency in this animal model too.

This experiment was carried out on three different test and control animals. All the animals showed a localization pattern which was identical both in the control group and in the group with cardiac insufficiency in each case.

3. Obtaining Neonatal Rat Cardiomyocytes

Primary cardiomyocytes were isolated from neonatal rats to carry out a hypertrophy experiment. The rats were from one to seven days old and were sacrificed by cervical dislocation. To isolate the cardiomyocytes, the ventricles of the contracting hearts were removed and dissociated using the “Neonatal Cardiomyocyte Isolation System” (Worthington Biochemicals Corporation, Lakewood, N.J.). The ventricles were for this purpose washed twice with Hank's balanced salt solution without calcium and magnesium (CMF HBBS), cut up with a scalpel until they had a size of about 1 mm³ and subjected to a cold trypsin treatment (2-10° C.) over night. The next day, the trypsin treatment was stopped by adding a trypsin inhibitor, and then a collagenase treatment was carried out at 37° C. for 45 minutes. The cells were dissociated by pipetting, passed through a “cell strainer” (70 μm) and centrifuged at 60×g twice for 5 min. The cell pellet was then taken up in 20 ml of conventional adhesion medium. Seeding took place at a density of 6×10⁴ cells/cm² on gelatin-coated (Sigma, Deisenhofen) tissue culture dishes or cover glasses. The next morning, the medium was removed by aspiration and, after washing with DMEM (conventional cell culture medium) twice, replaced by cultivation medium.

Adhesion medium: DMEM/M-199 (4/1); 10% horse serum; 5% fetal calf serum; 1 mM sodium pyruvate; penicillin, streptomycin, amphotericin B

Cultivation medium: DMEM/M-199 (4/1); 1 mM sodium pyruvate

4. Stimulation of Isolated Neonatal Cardiomyocytes

The cells were stimulated two to six hours after the medium was changed. This was done by treating the cardiomyocytes with various stimulants or combinations of stimulants (see Table 1) for 48 hours, followed by analysis. It was possible to observe the progress of a single stimulation on the basis of the morphological changes in the cells (hypertrophy). Besides the morphological changes, immunofluorescence analyses were also used to determine hypertrophy parameters (DCMAG-1 recruitment).

5. Immunofluorescence Analysis of Stimulated Neonatal Cardiomyocytes

For the immunofluorescence analysis, the stimulated cardiomyocytes were washed twice with cold PBS and fixed with 3% paraformaldehyde solution in PBS for 20 minutes. After washing again with cold PBS, the cells were incubated twice with 100 mM ammonium chloride in PBS, for 10 min each time, at room temperature. This was followed by a further washing step with cold PBS and incubation with 0.2% Triton-X 100 in PBS at room temperature for 5 min. Washing twice with 0.1% gelatin in PBS was followed by incubation with the first antibody at 37° C. in a “humidity chamber” known to the skilled worker. The first antibody (against the second domain of DCMAG-1) was diluted 1/500 in incubation solution (0.5% Tween-20; 0.5% BSA; in PBS). This was followed after one hour by three washing steps with PBS at room temperature for 5 min each time. The second antibody (obtained from goat, directed against rabbit, Cy3-coupled; Dianova, Hamburg) was diluted 1/200 in incubation solution and likewise incubated with the fixed cells at 37° C. for one hour. After three further washing steps with PBS at room temperature for 5 min each time, and a brief immersion in deionized water, the preparations were covered with a layer of Histosafe (Linaris, Wertheim-Bettingen) and applied to slides. Evaluation took place under a microscope (Axiovert 100S, Cy3 filter set, Zeiss, Göttingen).

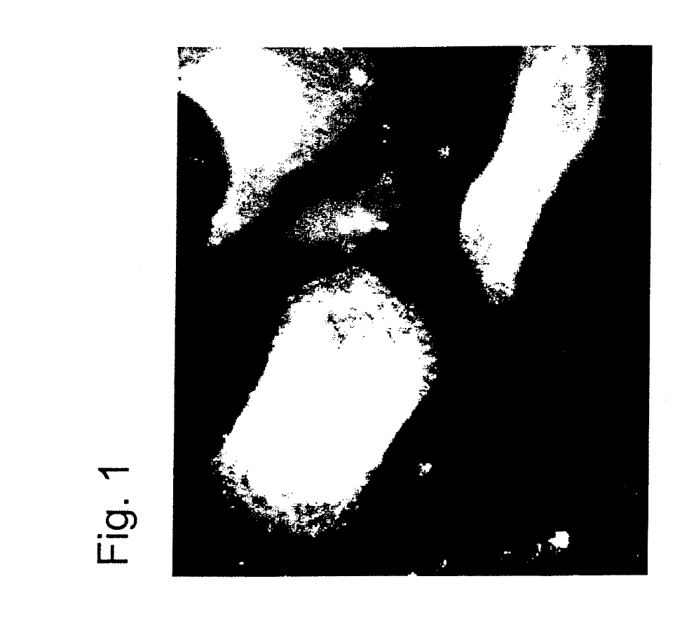

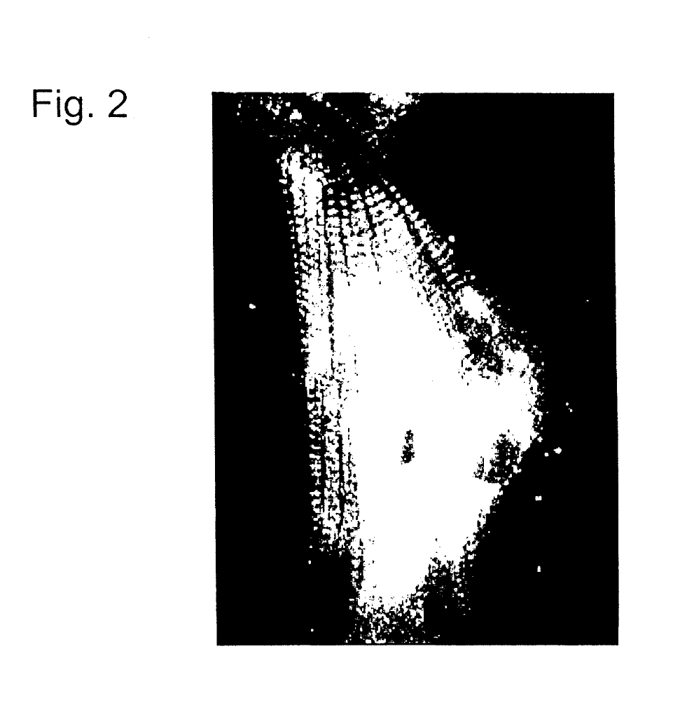

Unstimulated cardiomyocytes show a diffuse sarcoplasmic stain for DCMAG-1 (FIG. 1). DCMAG-1 is likewise distributed uniformly over the sarcoplasm for cells stimulated singly with PE or LIF, although the LIF-stimulated cells show an elongate shape. ET-1-stimulated cells show DCMAG-1 in filamentous structures. Cells doubly stimulated with ET-1 and PE show a weak sarcoplasmic pattern, whereas cells triply stimulated with ET-1, ISO and LIF show a distinctly visible striped pattern (FIG. 2).

Thus, for quantitative evaluation of these stimulation experiments, the recruitment of DCMAG-1 into the sarcomere was measured and categorized as follows:

(−) fewer than 2 cells per cover glass

(+) 2 to 5 cells per cover glass

(++) about 10% of the total cells

(+++) more than 10% of the total cells

TABLE 1

| 1 × stimulation | Sarcomere | 2 × stimulation | Sarcomere | 3 × stimulation | Sarcomere |
|---|---|---|---|---|---|
| none | (−) | | | | |
| PE | (−) | 0.5 × ET-1/PE | (−) | 0.5 × ET-1/LIF/ISO | (+) |
| LIF | (−) | 1.0 × ET-1/PE | (++) | 1.0 × ET-1/LIF/ISO | (++) |
| ET-1 | (−) | 2.0 × ET-1/PE | (++) | 1.5 × ET-1/LIF/ISO | (++) |
| ISO | (−) | 3.0 × ET-1/PE | (++) | 2.0 × ET-1/LIF/ISO | (+++) |
| IN | (−) | ET-1/0.5 × PE | (−) | 3.0 × ET-1/LIF/ISO | (+++) |
| 2 × PE | (−) | ET-1/1.0 × PE | (++) | 0.5 × ET-1/PE/ISO | (+) |
| 3 × PE | (−) | ET-1/2.0 × PE | (++) | 1.0 × ET-1/PE/ISO | (+) |
| 4 × PE | (−) | ET-1/3.0 × PE | (++) | 1.5 × ET-1/PE/ISO | (+) |
| 5 × PE | (−) | ET-1/LIF | (−) | 2.0 × ET-1/PE/ISO | (+) |
| 2 × ET-1 | (−) | ET-1/ISO | (−) | 3.0 × ET-1/PE/ISO | (+) |
| 3 × ET-1 | (−) | ET-1/IN | (−) | LIF/ISO/PE | (−) |
| 4 × ET-1 | (−) | IN/PE | (−) | IN/ISO/PE | (−) |
| 2 × ISO | (−) | IN/ISO | (−) | IN/LIF/ISO | (−) |
| 3 × ISO | (+) | IN/LIF | (−) | IN/ET-1/ISO | (−) |
| 4 × ISO | (+) | LIF/ISO | (+) | | |
| 5 × ISO | (+) | LIF/PE | (−) | | |
| 2 × LIF | (−) | PE/ISO | (−) | | |
| 2 × IN | (−) | 2 × PE/ISO | (−) | | |
| | | 2 × ISO/PE | (−) | | |
| | | 2 × ISO/LIF | (+) | | |
| | | 2 × ISO/ET-1 | (−) | | |
| | | 2 × ISO/IN | (−) | | |
| | | 2 × ET-1/ISO | (−) | | |
| | | 2 × LIF/ISO | (+) | | |

Note on Table 1: single dosage: PE:100 μM; LIF: 1 ng/ml; ET-1: 10 nM; ISO: 10 μM; IN: 100 nM

The results of the stimulation experiments, which are summarized in Table 1, show that a single stimulation brings about virtually no recruitment of DCMAG-1 into the sarcomere. There is merely a slight effect with high concentrations of ISO (see 1st column). The stimulation with two stimulants leads, in particular with the combination of ET-1 and PE, to a certain recruitment of DCMAG-1 into the sarcomere. Other combinations of two stimulants show only a slight or no effect (see 2nd column). The greatest recruitment of DCMAG-1 into the sarcomere is achieved by triple stimulation with ET-1, LIF and ISO. In all stimulation experiments showing a recruitment of DCMAG-1 into the sarcomere it was possible to observe localization of DCMAG-1 in the Z-band as well as in the M-line.

6. Stimulation of Isolated Neonatal Cardiomyocytes by Phorbol Ester

Besides the receptor stimulants mentioned above, it was surprisingly additionally found that incubation of neonatal rat cardiomyocytes with the PKC activator phorobol myristate-12,13 actetate (PMA, Sigma) brings about for the translocation of DCMAG-1 from the sarcoplasm to sarcomere structures. In these experiments, the cardiomyocytes were prepared as described above and seeded onto cover glasses. Stimulation with various concentrations of PMA was carried out for 48 hours, and the cells were fixed, stained as described above and investigated for DCMAG-1 translocation. The cells were visually classified and counted.

Counting of 6 independent experiments (±SEM) resulted in the following data for the localization of DCMAG-1:

TABLE 2

| Cardi- | % | % | % |
|---|---|---|---|
| omyocytes | dotted pattern | filamentous pattern | in the sarcomere |
| unstimulated | 74.9 ± 7.2 | 23.6 ± 6.2 | 0.1 ± 0.1 |
| LIF/ISO/ET-1 | 41.4 ± 4.2 | 38.4 ± 1.9 | 20.5 ± 3.8 |
| 1 nM PMA | 46.0 ± 2.0 | 20.0 ± 1.0 | 34.0 ± 3.0 |
| 100 nM PMA | 47.0 ± 4.0 | 13.0 ± 2.0 | 40.0 ± 2.0 |

The data listed in Table 2 show that the DCMAG-1 protein is translocated into the sarcomere even with the very small amount of 1 nM PMA. In addition, more cells show DCMAG-1 in the sarcomere after PMA stimulation than after triple stimulation with LIF/ISO/ET-1.

7. Localization of DCMAG-1 After PMA or Triple Stimulation

Since PMA brings about translocation of DCMAG-1 into the sarcomeres just like activation of three signal transduction pathways via their receptors, the sarcomeric structures into which DCMAG-1 was translocated with PMA or triple stimulation was investigated. Colocalization experiments were carried out for this purpose. Rat cardiomyocytes were seeded as described above on cover glasses, and correspondingly stimulated, fixed and stained. With the stains, anti-DCMAG-1 antibody (polyclonal) was mixed with either monoclonal α-actinin (Sigma, 1:500) or monoclonal anti-myosin (heavy chain, MHC, Sigma, 1:500). The secondary antibodies used were FITC-anti-mouse (1:250) and Texas Red-anti-rabbit (1:50; both from Dianova).

Evaluation took place with the aid of a fluorescence microscope, a Fuji-CCD camera and Aida software or with the aid of a confocal microscope (Pascal from Zeiss) and LSM software (Zeiss). It emerged from this that triple stimulation with ET-1/LIF/ISO resulted in a pattern of dots and stripes for the DCMAG-1 stain, with DCMAG-1 being arranged like strings of beads along the sarcomeres. Compared with the actinin stain, which likewise shows a pattern of dots and stripes, there are twice as many dots/stripes for DCMAG-1 as for actinin, with colocalization of every second dot/stripe. Since actinin specifically stains the Z-band, after this triple stimulation DCMAG-1 is to be found in the Z-band and in the M-line.

Stimulation of cardiomyocytes with PMA on the other hand brought about an alteration in the color pattern. Double staining with α-actinin and DCMAG-1 led to alternately red and green transverse stripes, which means that there was no colocalization of α-actinin and DCMAG-1 in this case. Double staining of MHC and DCMAG-1 led to a picture which can be described as a sequence of a black line, a green band, a yellow line, a green band and finally another black line. Units of this type were arranged like strings of beads and permeated the sarcoplasm. This shows that DCMAG-1 colocalizes with the M-line after PMA stimulation and moreover is to be found in the middle of the M-line in each case. Thus, after stimulation with PMA, DCMAG-1 translocates into the M-line. (Evaluation with confocal microscope: Axiovert 100 and LSM 410 software from Zeiss).

DCMAG-1 may accordingly be found in different structures in the sarcomere, depending on the stimulant which acts.

8. Measurement of the Effect of Inhibitors on DCMAG-1 Translocation in the Immunofluorescence Test in Cardiomyocytes from the Neonatal Rat

Neonatal rat cardiomyocytes were prepared as in Example 3 and seeded in a density of 1×10⁵ cells per 1.5 cm well (reaction chamber in a cell culture dish). The cell culture dishes contained 1.5 cm cover glasses (Schubert und Weiβ) coated with 1% gelatin solution. The cells were incubated after 24 hours with DMEM and subsequently in maintenance medium with or without stimulus (LIF/ISO and ET-1, concentration as above for single dosage) for 48 hours.

In order to determine the signal transduction pathways required for translocation of DCMAG-1, the stimulated cells were incubated with inhibitors, namely 30 μM LY294002 (Sigma), 50 μM SB 203580 (Sigma), 15 nM Go 6976 (Alexis) or 50 μM PD98059 (NEB) for 48 h (after 24 h, the medium and inhibitors were renewed because the activity of the inhibitors was limited to 24 h in aqueous solution).

After 48 h, the cells were fixed with 4% paraformaldehyde, permeabilized with 0.2% Triton-X100 and stained with anti-DCMAG (polyclonal, own production, 1:500) or α-actinin (as control, Sigma, 1:500) and visualized with anti-mouse or anti-rabbit Cy3 (Jackson Labs, USA) in immunofluorescence. In order to measure the effect of the inhibitors, the cells were visually classified and counted.

Counting of 6 independent experiments (±SEM) resulted in the following data for the localization of DCMAG-1:

TABLE 3

| | % dotted | % filamentous | % in |
|---|---|---|---|
| Cardiomyocytes | pattern | pattern | Z-bands |
| unstimulated | 74.9 ± 7.2 | 23.6 ± 6.2 | 0.1 ± 0.1 |
| stimulated | 41.4 ± 4.2 | 38.4 ± 1.9 | 20.5 ± 3.8 |
| stimulated + LY | 46.6 ± 5.3 | 41.2 ± 4.1 | 11.3 ± 2.0 |
| stimulated + PD | 58.5 ± 10.7 | 31.6 ± 7.4 | 10.1 ± 3.4 |
| stimulated + Gö | 29.0 ± 2.1 | 49.0 ± 0.0 | 22.0 ± 2.1 |
| stimulated + SB | 22.5 ± 5.3 | 35.5 ± 0.3 | 41.0 ± 4.9 |
| stimulated + LY + PD | 83.9 ± 7.2 | 15.7 ± 7.5 | 0.4 ± 0.2 |
| stimulated + LY + Gö | 71.5 ± 0.4 | 28.0 ± 1.4 | 1.0 ± 0.7 |
| stimulated + LY + SB | 65.0 ± 4.0 | 28.0 ± 4.0 | 6.0 ± 2.0 |
| stimulated + SB + PD | 90.0 ± 5.6 | 9.3 ± 4.8 | 1.0 ± 0.7 |

stimulated = stimulation with LIF, ISO and ET-1
LY = addition of LY294002 (Sigma)
SB = addition of SB 203580 (Sigma)
Gö = addition of Gö 6976 (Alexis)
PD = addition of PD98059 (NEB)

Total number of counted cells=5092;

The inhibition experiments, summarized in Table 3, show that various substances are suitable for reducing the translocation of DCMAG-1 into the sarcomere (see last column for LY, PD, Gö) and substance combinations for almost completely preventing the translocation (LY+PD, LY+Gö, SB+PD). This test system is therefore suitable for looking for active ingredients or active ingredient combinations for reducing or preventing the translocation of DCMAG-1 into the sarcomere.

1. A pathologically modified myocardial cell which can be produced from healthy cardiac tissue and/or at least one healthy myocardial cell by a method comprising the steps:
(a) provision or isolation of at least one healthy myocardial cell;
(b) stimulation of the isolated myocardial cell by suitable hormones, hormone analogs and/or cytokines.

2. A pathologically modified myocardial cell as claimed in claim 1, characterized in that the healthy cardiac tissue is derived from birds, in particular from chickens, or from mammals, in particular from humans, rodents, preferably rats, or rabbits.

3. A pathologically modified myocardial cell as claimed in claim 1 or 2, characterized in that the myocardial cell is stimulated essentially simultaneously by at least two, in particular three, different hormones, hormone analogs and/or cytokines.

4. A pathologically modified myocardial cell as claimed in any of claims 1 to 3, characterized in that hormones, hormone analogs and/or cytokines are selected from ET-1, ISO, PE and/or LIF.

5. A pathologically modified myocardial cell as claimed in any of claims 1 to 4, characterized in that the essentially simultaneous stimulation is effected by various hormones, hormone analogs and/or cytokines via at least partly different levels of the signal transduction cascades of the cell.

6. A pathologically modified myocardial cell as claimed in any of claims 1 to 5, characterized in that the essentially simultaneous stimulation is effected via at least two, in particular at least three, receptors, preferably via a G_{q}-coupled receptor, in particular an ET-1 receptor, and/or via a β-adrenergic receptor, in particular a receptor which can be stimulated by ISO, and/or via a cytokine receptor, in particular an LIF receptor (GP130).

7. A pathologically modified myocardial cell as claimed in any of claims 1 to 6, characterized in that the essentially simultaneous stimulation of the signal transduction cascade is effected at a level subject to receptor stimulation, preferably by phorbol esters.

8. A method for producing a pathologically modified myocardial cell from healthy cardiac tissue and/or at least one healthy myocardial cell as claimed in any of claims 1 to 7, characterized in that the method comprises the following steps:
(i) provision or isolation of at least one healthy myocardial cell;
(ii) stimulation of the isolated myocardial cell by suitable hormones, hormone analogs and/or cytokines; and where appropriate
(iii) detection of the at least one pathologically modified myocardial cell by determination of the localization of at least one signal molecule, preferably at least one protein, in the sarcomere.

9. A method for producing a pathologically modified myocardial cell as claimed in claim 8, characterized in that the localization of said protein in step (iii) takes place at the single-cell level.

10. A method for producing a pathologically modified myocardial cell as claimed in claim 8 or 9, characterized in that the localization of said protein in step (iii) is determined in the Z-band and/or in the M-line of the sarcomere.

11. A method for producing a pathologically modified myocardial cell as claimed in any of claims 8 to 10, characterized in that said protein in step (iii) is associated with structures of the sarcomere, in particular the M-line or the Z-band, and leads to characteristic modifications of sarcomere proteins, in particular M-line proteins or Z-band proteins, preferably tyrosine, serine and/or threonine phosphorylations.

12. A method for producing a pathologically modified myocardial cell as claimed in any of claims 8 to 11, characterized in that said protein in step (iii) has structural features of tropomodulin, in particular a tropomyosin binding domain.

13. A method for producing a pathologically modified myocardial cell as claimed in any of claims 8 to 12, characterized in that said protein in step (iii) has the amino acid sequence shown in SEQ ID NO: 1 or a functional variant thereof, in particular at least one mutation and/or deletion.

14. A method for producing a pathologically modified myocardial cell as claimed in claim 13, characterized in that said functional variant has a homology with SEQ ID NO: 1 of at least about 50%, in particular of at least about 60%, especially of at least about 70%.

15. A method for producing a pathologically modified myocardial cell as claimed in claim 13 or 14, characterized in that the amino acid sequence shown in SEQ ID NO: 1 or a functional variant thereof is encoded by a nucleic acid, preferably by a DNA or RNA, particularly preferably by a cDNA.

16. A method for the detection or for the identification of one or more substances acting on the heart, characterized in that the method comprises the following steps:
(i) provision or isolation of at least one myocardial cell as claimed in any of claims 1 to 7;
(ii) contacting of the myocardial cell with one or more test substances; and
(iii) detection or identification of one or more substances acting on the heart through determination of the localization of at least one signal molecule, preferably at least one protein, in the sarcomere.

17. A method as claimed in claim 16, characterized in that the myocardial cell is a pathologically modified myocardial cell as claimed in any of claims 1 to 7.

18. A method as claimed in claim 16 or 17, characterized in that said test substance is a pharmaceutically effective substance.

19. A method as claimed in claim 16 or 17, characterized in that said test substance is a toxic substance.

20. A method as claimed in any of claims 16 to 19, characterized in that said test substance is a low molecular weight, inorganic or organic molecule, an expressible nucleic acid, preferably a protein, a natural or synthetic peptide or a complex thereof, which reduces and/or essentially prevents localization of the signal molecule into the sarcomere, in particular into the M-line or the Z-band.

21. A method as claimed in any of claims 16 to 19, characterized in that said test substance is a low molecular weight, inorganic or organic molecule, an expressible nucleic acid, preferably a protein, a natural or synthetic peptide or a complex thereof, which favors and/or essentially brings about localization of the signal molecule into the sarcomere, in particular into the M-line or the Z-band.

22. The use of a pathologically modified myocardial cell as claimed in any of claims 1 to 7 for the detection or for the identification of one or more substances acting on the heart.
